(19) United States
(12) Patent Application Publication
(10) Pub. No.: US 2006/0079819 A1
(43) Pub. Date: Apr. 13, 2006

(54) Orthopedic splint

(75) Inventors: John C. Evans, Charlotte, NC (US); Shitij Chabba, Charlotte, NC (US); Martin O'Hara, Charlotte, NC (US)

Correspondence Address: Adams Evans P.A.;2180 Two Wachovia Center, 301 S. Tryon Street, Charlotte, NC, 28282, US

(73) Assignee: BSN Medical, Inc., Wilmington, DE (US)

(21) Appl. No.: 11/093,057

(22) Filed: Mar. 29, 2005

Related U.S. Application Data
(60) Provisional application No. 60/618,250, filed on Oct. 13, 2004.

(51) Int. Cl. A61F 5/00

(52) U.S. Cl. 602/6; 602/5; 602/8

(57) ABSTRACT

An orthopedic splint utilizing a polyurethane-based chemical system encapsulated in an envelope that hardens to form a splint. The orthopedic splint includes an envelope for containing a first component and a second component of maintained separate from each other within the envelope. A hardened splint is formed by causing the first component and the second component to mix in the envelope.

### TECHNICAL FIELD AND BACKGROUND OF THE INVENTION

This application claims the benefit of Provisional Application No. 60/618,250 filed on Oct. 13, 2004.

The present invention relates to orthopedic splints. In particular, this invention relates to orthopedic splinting materials and methods utilizing a polyurethane-based chemical system encapsulated in an envelope that hardens to form a splint.

Medical bandages for use in the treatment of injuries, such as broken bones requiring immobilization of a body member, are generally formed from a strip of fabric or scrim material impregnated with a substance which hardens into a rigid structure after the strip has been wrapped around the body member. The hardening substance traditionally used in carrying out this procedure is plaster-of-paris.

Conventional practice has been to fabricate a cast or splint upon an injured limb by initially applying to the limb a protective covering of a cotton fabric or the like, and then overwrapping the covering and limb with a woven cloth impregnated with plaster-of-paris which has been wetted by dipping in water immediately prior to application. This practice is still in widespread use but is messy and time-consuming. Several components are required and considerable skill is necessary.

In order to alleviate the above-recited disadvantages of the conventional application procedure for plaster-of-paris casts and splints, unitary splinting materials have been devised and are disclosed in, for example, U.S. Pat. Nos. 3,900,024, 3,923,049, and 4,235,228. All of these patents describe a padding material with a plurality of layers of plaster-of-paris impregnated cloth. Such unitary splinting materials are not as messy and can be applied more quickly but still suffer from a number of disadvantages inherent in plaster-of-paris cast materials. All plaster-of-paris splints have a relatively low strength to weight ratio which results in a finished splint which is very heavy and bulky. Plaster-of-paris splints are slow to harden, requiring 24 to 72 hours to reach maximum strength. Since plaster-of-paris breaks down in water, bathing and showering are difficult. Even if wetting due to these causes can be avoided, perspiration over an extended period of time can break down the plaster-of-paris and create a significant problem with odor and itching.

A significant advance in the art of casting and splinting is disclosed in U.S. Pat. Nos. 4,411,262 and 4,502,479. The splinting materials disclosed in these patents comprise a flexible fabric impregnated with a moisture-curing resin enclosed in a moisture-free, moisture-impervious package. Compared to plaster-of-paris, these products are extremely lightweight, have a very high strength-to-weight ratio and can be made relatively porous, permitting a flow of air through the splinting material. No provision is made for reclosing the package, so that the entire material must be very quickly used after removal from the package since such moisture-curing resins will cure in a relatively short period of time due merely to contact with atmospheric moisture.

A further development in moisture-curable resin casts and splints is disclosed in U.S. Pat. No. 4,770,299. The unitary splinting system disclosed in this patent comprises a moisture-curing resin casting material, together with a moisture-impervious package with means for resealing the package against entry of moisture after a desired length of bandaging product has been removed for use. The sealing of the package prevents the hardening of the bandaging product remaining in the moisture-impervious package.

From the above discussion, it can be seen that both the conventional plaster-of-paris casting method and the more recent moisture-curable resin casting method possess both advantages and disadvantages. On the one hand, plaster-of-paris casts are bulky, heavy and difficult to apply whereas moisture-curable resin casts are lightweight, durable and relatively easy to apply. Plaster-of-paris can be very easily stored and used as needed since it has a relatively long shelf life so long as it is not completely wetted. On the other hand, the moisture-curable resins are very sensitive to the presence of even minute amounts of moisture which requires that either the materials be packaged in a wide variety of different shapes and sizes or sealed against moisture. In addition, both plaster-of-paris casts and moisture-curable resin casts require water to harden.

This invention combines the advantages of both plaster-of-paris and moisture-curable resin systems while avoiding their respective disadvantages. This is accomplished by providing a unitary splinting system which has the characteristics of the moisture-curable resin cast of U.S. Pat. No. 4,770,299, but eliminates the water requirement for hardening. The unitary splinting system is provided with the use of a polyurethane-based chemical system, together with an envelope for containing the chemical system. In this manner, the hardening of the splinting product can be accomplished without the need of an external curing agent such as water.

### SUMMARY OF THE INVENTION

Therefore, it is an object of the invention to provide a flexible and conformable orthopedic splint that hardens without exposure to water.

It is another object of the invention to provide an orthopedic splint that hardens upon mixing of a pre-polymer and an activator or curing agent contained in an envelope.

It is another object of the invention to provide an orthopedic splint that has an envelope designed to maintain the pre-polymer and polyol in a separate condition until ready for use.

It is another object of the invention to provide an orthopedic splint that is clean and does not require use of gloves to avoid contact with chemicals.

These and other objects of the present invention are achieved in the preferred embodiments disclosed below by providing an orthopedic splint that includes an envelope for containing a first component and a second component of a polymer system. A seal is provided for separating the first component from the second component within the envelope. Breaking of the seal allows the first component and the second component to mix in the envelope, thereby forming a hardened splint.

According to another preferred embodiment of the invention, the envelope has a length sufficient to extend a length of a body part and a width sufficient to wrap approximately 50% of a circumference of the body part for immobilizing and allowing the body part to swell.

According to another preferred embodiment of the invention, the envelope is constructed of a material selected from the group consisting of aluminum foil, polyester, polypropylene, polyurethane, nylon, PCTFE, PVDC, metallised polyester, metallised polypropylene, PTFE, polyethylene, polyvinyl chloride (PVC), ethylvinyl alcohol (EVA), and/or a composite laminate formed using two or more of these materials.

According to another preferred embodiment of the invention, the envelope includes a protective layer disposed on an outside surface of the envelope for being positioned between a body part and the splint and providing protection to a patient.

According to another preferred embodiment of the invention, the first component is a pre-polymer and the second component is an activator.

According to another preferred embodiment of the invention, the orthopedic splint further includes a substrate contained within the envelope, the substrate being impregnated with a respective one of the first and second components.

According to another preferred embodiment of the invention, an orthopedic splint is provided that includes an envelope for containing a polymer system, and at least one smaller envelope. The polymer system includes a first component and a second component for mixing with the first component to form a hardened splint. The smaller envelope is contained within the other envelope for containing a respective one of the first and second components, thereby separating the first and second components within the envelope in a pre-mixed state. The smaller envelope is ruptured to allow the first and second components to mix within the envelope, forming the hardened splint.

According to another preferred embodiment of the invention, the first component is a pre-polymer selected from the group consisting of polyurethane, epoxy, polyolefin, polyester, silicone, and polyurea.

According to another preferred embodiment of the invention, the second component is an activator selected from the group consisting of water, hydrogels, amines, and polyols.

According to another preferred embodiment of the invention, the smaller envelope is formed from the envelope and is defined by and separated from the envelope by a seal.

According to another preferred embodiment of the invention, the smaller envelope is a separate structure inserted into the envelope.

According to another preferred embodiment of the invention, the smaller envelope contains the first component and the envelope contains the second component.

According to another preferred embodiment of the invention, the smaller envelope contains the second component and the envelope contains the first component.

According to another preferred embodiment of the invention, the orthopedic splint further includes a substrate contained within the envelope. The substrate is impregnated with a respective one of the first and second components.

According to another preferred embodiment of the invention, the substrate is a fabric constructed of any suitable organic or inorganic fiber, preferably selected from the group consisting of polyethylene, polypropylene, para-aramid, and polyester.

According to another preferred embodiment of the invention, an orthopedic splint is provided that includes an envelope for containing a polymer system, a substrate, and at least one burstable bubble. The polymer system includes a pre-polymer and a polyol for mixing with the pre-polymer to form a hardened splint. The substrate is contained within the envelope and impregnated with the pre-polymer. The burstable bubble is contained within the envelope and contains the activator or curing agent, thereby separating the pre-polymer and the activator within the envelope in an unmixed state, wherein the at least one burstable bubble is ruptured to allow the activator to mix with the pre-polymer contained within the substrate, forming the hardened splint.

According to another preferred embodiment of the invention, the orthopedic splint further includes seals to hold the burstable bubble in a desired position within the envelope.

According to another preferred embodiment of the invention, at least one burstable bubble is constructed of a material selected from the group consisting of polyethylene, aluminum foil, PCTFE, polyester, PTFE, nylon, polyester, polypropylene, metallized film, EVA, and PVC.

According to another preferred embodiment of the invention, the splint has a predetermined pre-cut length for being applied to a body part.

According to another preferred embodiment of the invention, the splint is in roll form for being dispensed in lengths suitable for a given medical use.

According to another preferred embodiment of the invention, a method of constructing an orthopedic splint includes the steps of providing an initially flexible and conformable envelope, and providing first and second compartments in the envelope. A first component is contained in the first compartment and a second component is contained in the second compartment that when mixed, cause the mixture to harden into the splint.

According to another preferred embodiment of the invention, the method further includes the step of applying a protective layer on an outside surface of the envelope.

According to another preferred embodiment of the invention, a method of immobilizing a body part includes the steps of providing an orthopedic splint including an initially flexible and conformable envelope containing a first component and a second component of a two component polymer system, and a seal for separating the first component from the second component within the envelope. Applying pressure to the orthopedic splint, thereby rupturing the seal and allowing the first component and the second component to mix. Placing the splint into engagement with the body part and into a position whereby the body part is supported in a desired position, and securing the splint to the body part in a closely-conforming configuration for a period of time sufficient to allow the splint to harden.

According to another preferred embodiment of the invention, the method further includes the step of massaging opposing ends of the splint to provide mixing of the first and second components.

According to another preferred embodiment of the invention, the method further includes the step of molding the splint to the body part while the splint is flexible.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 shows an orthopedic splint of the present invention being formed to fit the contour of a body member;

FIG. 2 shows the orthopedic splint of FIG. 1 with a protective layer;

FIG. 3 is a perspective view of the hardening orthopedic splint of FIG. 1 being secured into place on a body member by means of a securing strip;

FIG. 4A is a perspective view of a first embodiment of the orthopedic splint of FIG. 1 in a non-mixed state;

FIG. 4B is another perspective view of the orthopedic splint of FIG. 3A in a mixing state;

FIG. 4C is another perspective view of the orthopedic splint of FIG. 3A in a mixed state;

FIG. 5A is a perspective view of a second embodiment of the orthopedic splint of FIG. 1 in a non-mixed state;

FIG. 5B is another perspective view of the orthopedic splint of FIG. 4A in a mixing state;

FIG. 5C is another perspective view of the orthopedic splint of FIG. 4A in a mixed state;

FIG. 6A is a perspective view of a third embodiment of the orthopedic splint of FIG. 1 in a non-mixed state;

FIG. 6B is another perspective view of the orthopedic splint of FIG. 5A in a mixing state;

FIG. 6C is another perspective view of the orthopedic splint of FIG. 5A in a mixed state;

FIG. 7A is a perspective view of a fourth embodiment of the orthopedic splint of FIG. 1 in a non-mixed state;

FIG. 7B is another perspective view of the orthopedic splint of FIG. 6A in a mixing state;

FIG. 7C is another perspective view of the orthopedic splint of FIG. 6A in a mixed state;

FIG. 8A is a perspective view of a fifth embodiment of the orthopedic splint of FIG. 1 in a non-mixed state;

FIG. 8B is another perspective view of the orthopedic splint of FIG. 7A in a mixing state;

FIG. 8C is another perspective view of the orthopedic splint of FIG. 7A in a mixed state;

FIG. 9A is a perspective view of a sixth embodiment of the orthopedic splint of FIG. 1;

FIG. 9B is a cross-sectional view of the splint of FIG. 9A;

FIG. 9C is another perspective view of the splint of FIG. 9A showing a bubble being ruptured;

FIG. 9D is a cross-sectional view of the splint of FIG. 9A before pressure is applied to the bubble;

FIG. 9E is a cross-sectional view of the splint of FIG. 9A showing pressure being applied to the bubble;

FIG. 9F is a cross-sectional view of the splint of FIG. 9A showing the bubble rupturing;

FIG. 9G is a cross-sectional view of the splint of FIG. 9A in a mixing state;

FIG. 9H is a another perspective view of the splint of FIG. 9A in a mixing state;

FIG. 10A is a perspective view of a seventh embodiment of the orthopedic splint of FIG. 1;

FIG. 10B is an exploded view of the splint of FIG. 10A;

FIG. 10C is another perspective view of the splint of FIG. 10A showing a bubble being ruptured;

FIG. 10D is a cross-sectional view of the splint of FIG. 10A before pressure is applied to the bubble;

FIG. 10E is a cross-sectional view of the splint of FIG. 10A showing pressure being applied to the bubble;

FIG. 10F is a cross-sectional view of the splint of FIG. 10A showing the bubble rupturing;

FIG. 10G is a cross-sectional view of the splint of FIG. 10A in a mixing state;

FIG. 10H is a another perspective view of the splint of FIG. 10A in a mixing state; and

FIG. 11 shows the splint of FIG. 10A rolled up in a container.

### DESCRIPTION OF THE PREFERRED EMBODIMENT AND BEST MODE

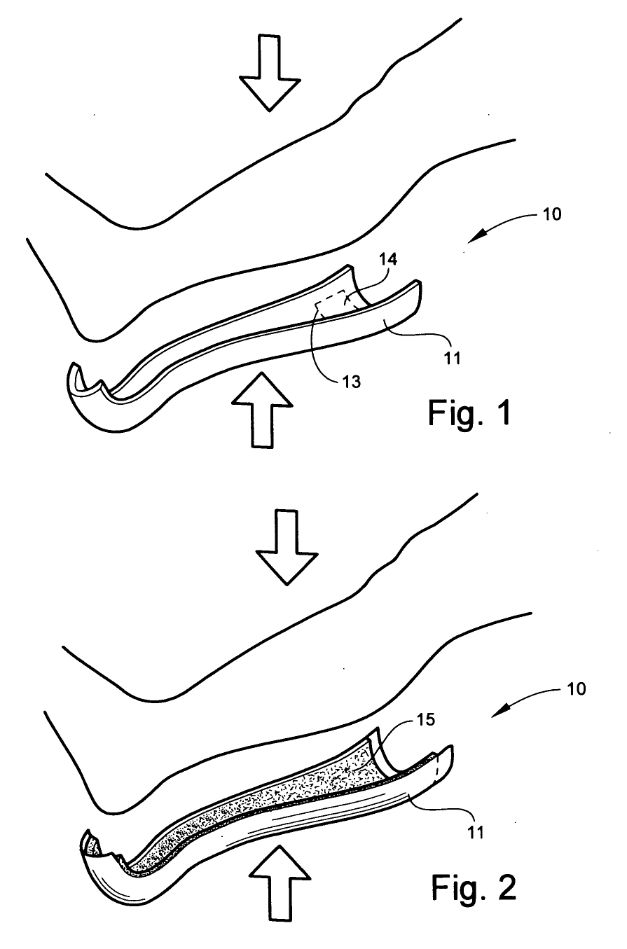

Referring now specifically to the drawings, a waterless orthopedic splint according to an embodiment of the present invention is illustrated in FIGS. 1-3 and shown generally at reference numeral 10.

The splint 10 is formed of an outer elongate envelope 11 in which is contained a polyurethane polymer system, described in detail below. The splint 10 is suitable for use on a body part, human or animal.

The envelope 11 has a length sufficient to extend the length of the body part and width sufficient to wrap approximately 50% of the circumferential body part thereby immobilizing the limb or body part, but also allow for any swelling that may occur as part of a recent injury. The splint 10, while in the process of setting, can be molded around the fracture or injury and held in place with a securing strip 12 such as a medical wrap, bandage or hook and loop fasteners that will keep the splint 10 in position.

The envelope 11 can be constructed of any flexible, moldable material that provides a moisture barrier, good puncture resistance, and is sealable. The envelope material can be a laminate or a single sheet. Typical materials that can be used in the construction of the envelope 11 are aluminum foil, polyester, polypropylene, polyurethane, nylon, PCTFE, PVDC, metallised polyester, metallised polypropylene, polyethlyene, PVC, EVA, or a laminate comprising two or more of these materials.

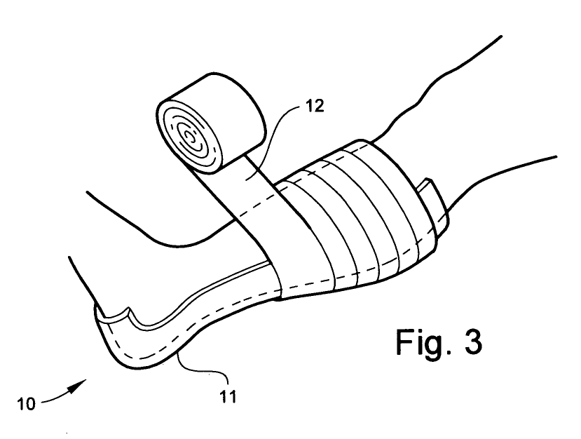

The envelope 11 can be used with or without a padding or protective layer 15, as illustrated in FIGS. 1 and 2. The padding can be constructed of polypropylene, such as the tubular material described in U.S. Pat. No. 4,770,299, or any other suitable material.

The envelope can also be used with a separate protective layer positioned between the envelope 11 and the patient, or a cover applied only to one side, for example, a non-woven cover having a thickness of 1 to 2 millimeters. The covered side is placed next to the skin. The padding layer can be used on one or both sides of the out envelope.

The splint 10 incorporates a two component polyurethane polymer system. The first component is a pre-polymer and the second component is an activator such as water, amines, and polyols. Both the pre-polymer and the activator are contained within the envelope 11. The pre-polymer and activator are kept separate inside the envelope 11 by a separation means until activation is required, at which time a reaction takes place forming the liquid into a hard compound. The separation means includes any suitable method or device, such as a weakness line, a seal, or a burstable bubble, for maintaining the pre-polymer and polyol separate and in proximate relation to each other until activation.

The activator system contained within the envelope 11 can be based on a multifunctional polyol, a polyether type or polyester type polyol, a copolymer polyol or primary or secondary amine/diamine or hydrogel. The pre-polymer usually has an excess of either polyol or isocyanate depending on the desired properties. The isocyanate can be an aliphatic or aromatic or cycloaliphatic isocyanate such as methylene diphenyl diisocyanate (MDI) or Toluene diisocyanate (TDI) or isophorone diisocyanate (IPDI).

The pre-polymer and activator can be selected to achieve the desired structural density forming either an open cell foam, closed cell foam, reticulated cell foam, micro-cellular foam or solid rigid structure. The hardness of the structure can be adjusted by manipulating the chemistry of the compound resulting in a final splint density of at least 5 pounds per cubic foot.

The pre-polymer should be selected from a wide group of polymers that include but is not limited to, polyurethane, epoxy, polyolefin, polyester, polyurea, and silicone, with the intention of creating a final orthopedic splint that is tough and not brittle so that it can be used for weight bearing applications, for example, a posterior leg splint.

A typical two-component polyurethane polymer system is set forth below:

| IPDI/blend of polycaprolactone diol | pre-polymer | 29.58 g |
|---|---|---|
| Clearlink ® 1000 | activator | 12.42 g |
| Isocyanate index | | 100 |

An example of the pre-polymer, expressed in parts by weight, used above is as follows:

| | | |
|---|---|---|
| Tone ™ 32C8 | polyol | 242.7 |
| Tone ™ 32B8 | polyol | 106.6 |
| PCD-982 | polyol | 129.6 |
| IPDI | | 320.8 |
| NCO % | | 10% |

The splint system is designed to ensure a fast mixing of the components encapsulated in the envelope 11. The mixing time is less than 5 minutes, preferably between 1 to 3 minutes, and more preferably between 15 to 30 seconds. The two components upon mixing should allow a working time before setting of 0.5 to 10 minutes, preferably 45 to 60 seconds. The initial setting time of the compound to support the fracture should be 1 to 12 minutes, preferably 1.5 to 3.0 minutes. The final set time sufficient to fully immobilize the fracture or injury should be less than 30 minutes, preferably not to exceed 10 minutes.

An important feature of the splint system is that the exothermic reaction does not exceed 40 degrees Celsius and should preferably be less than 35 degrees Celsius, such that the splint is comfortable for patients with sensitive skin.

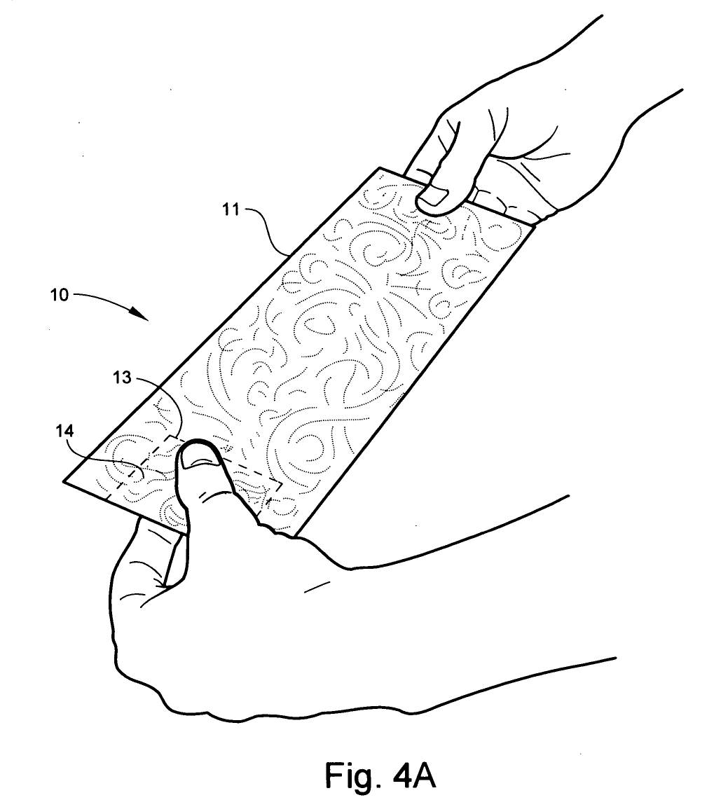

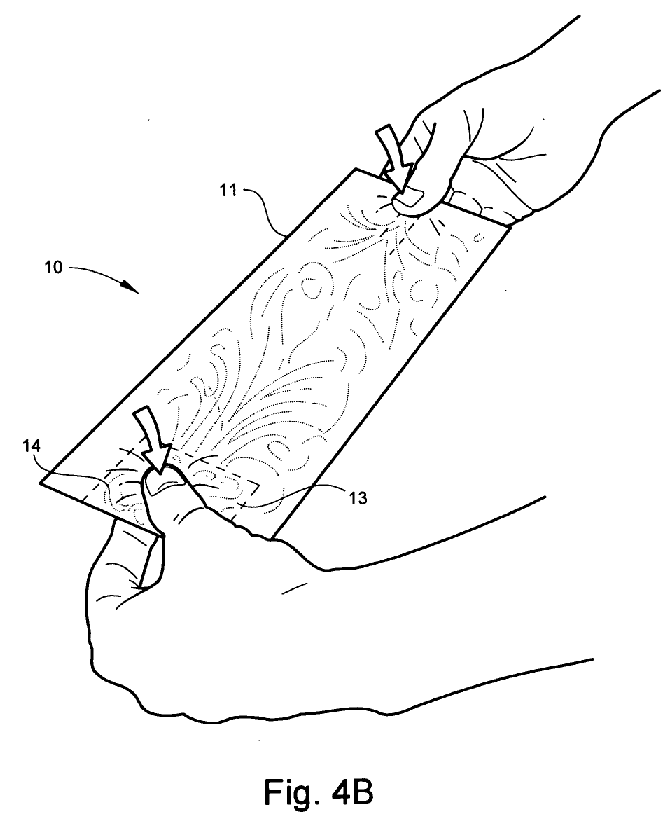

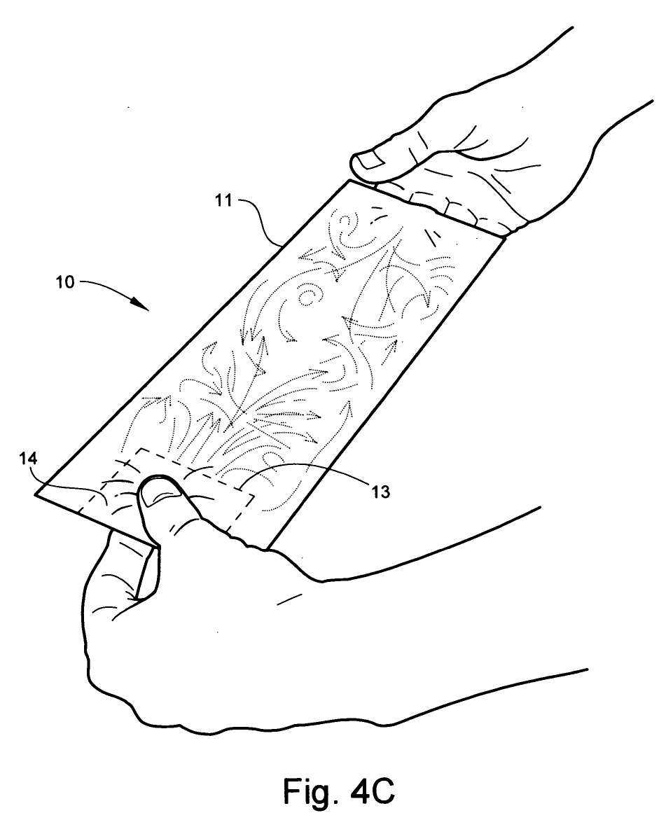

The activator and pre-polymer are separated within the envelope 11, as illustrated in FIGS. 4-10, to prevent mixing of the polymer system and a consequent reaction between the pre-polymer and the polyol prior to the time of use. The splint 10 is activated by applying manual pressure to the envelope 11 and breaking a seal 13 that divides the activator from the pre-polymer, allowing the two components to mix together. The seal 13 is shown as a dotted line for illustration purposes only. The dotted line does not represent the structure of the seal 13.

Various embodiments of the splint 10 may be used to prevent mixing of the pre-polymer and activator. Seven embodiments of the splint 10 are discussed below, however, any suitable design that keeps the two components separate until activation is required may be constructed. In each of the embodiments, the splint 10 goes through three phases. In phase one, no mixture of the pre-polymer and activator has occurred. In phase two, the pre-polymer and the polyol have begun mixing. In phase three, a complete mixture of the pre-polymer and polyol has been achieved.

In a first embodiment, the pre-polymer is contained in a single, smaller envelope 14, and the activator is contained within the envelope 11, as illustrated in FIG. 4A. When the seal 13 of the envelope 14 is broken, as shown in FIG. 4B, the pre-polymer is mixed in the envelope 11 with the polyol until complete mixture has been obtained, FIG. 4C, causing a reaction and hardening of the splint.

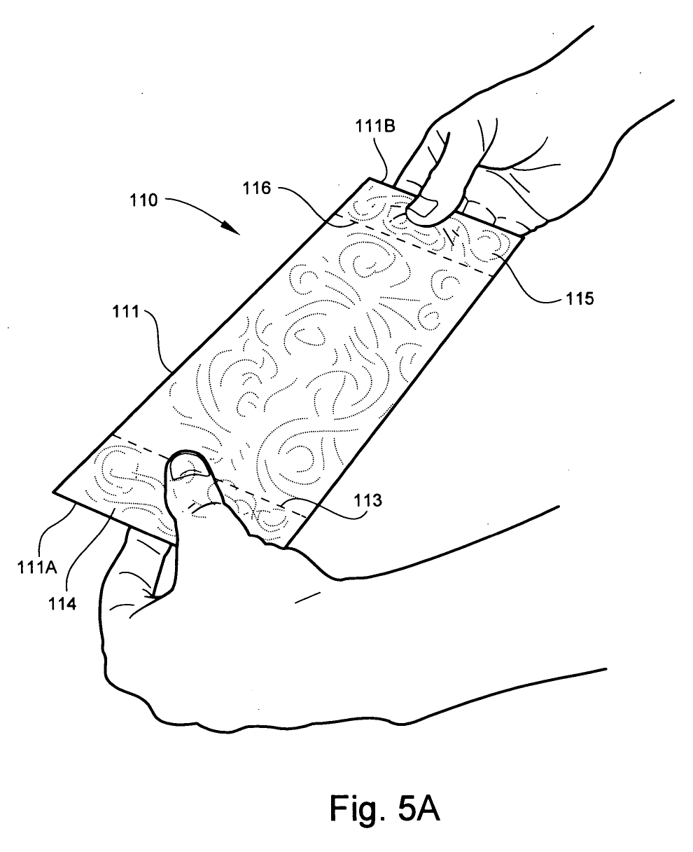

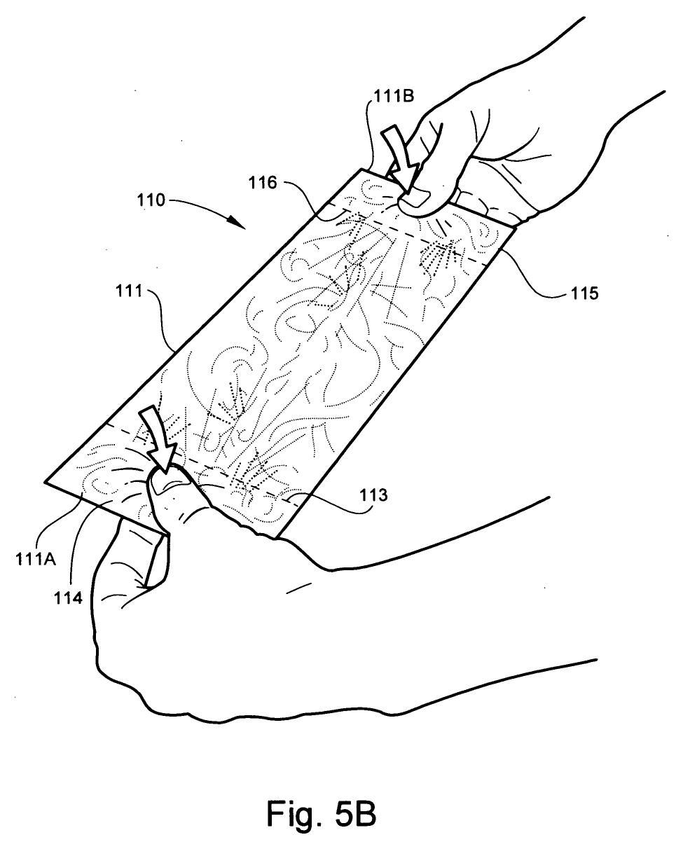

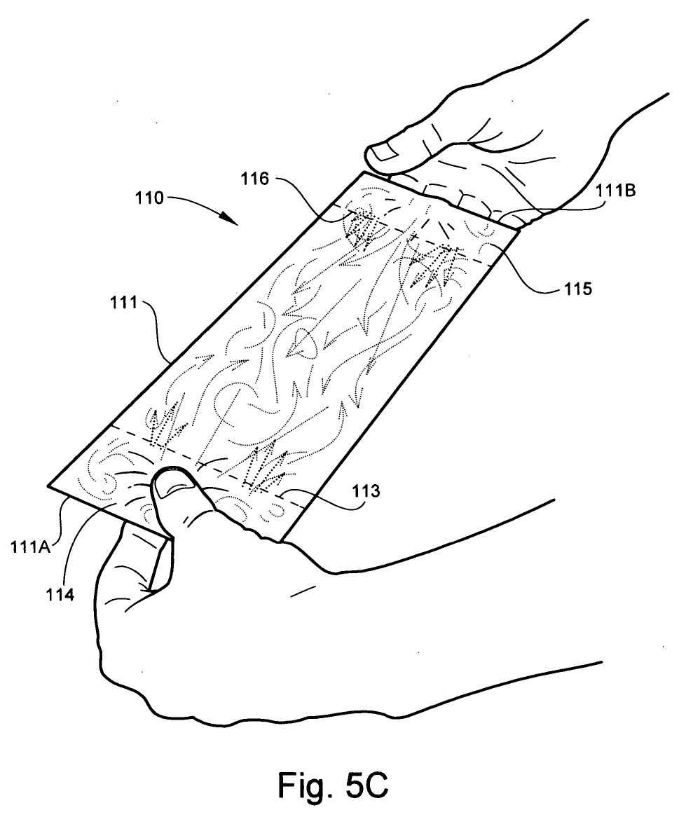

A splint 110 according to a second embodiment is shown in FIGS. 5A-5C. The splint 110 includes two smaller envelopes 114 and 115 that are positioned on opposing ends 111A and 111B of an envelope 111, and are used to contain the pre-polymer. When seals 113 and 116 of the two envelopes 114 and 115 are broken, the pre-polymer is released into the envelope 111 where it is mixed with the activator.

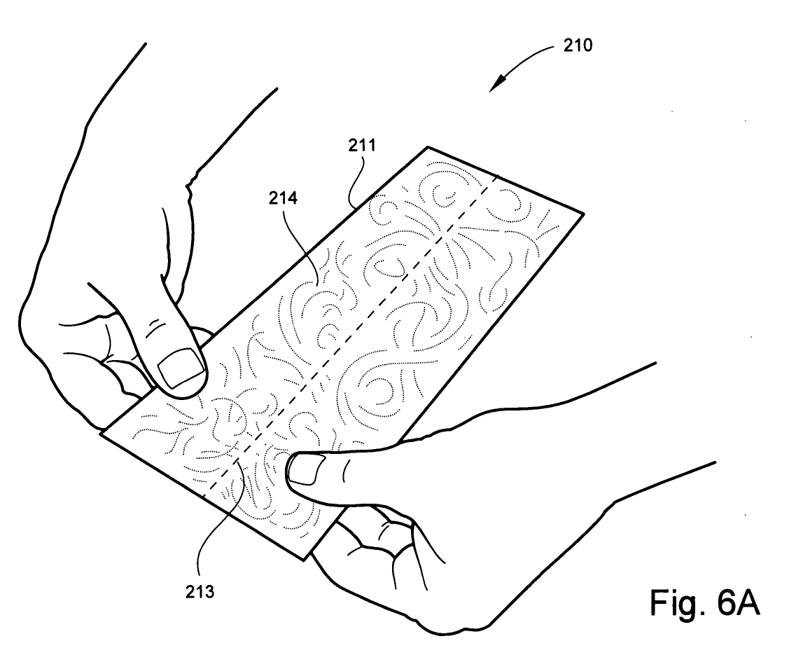

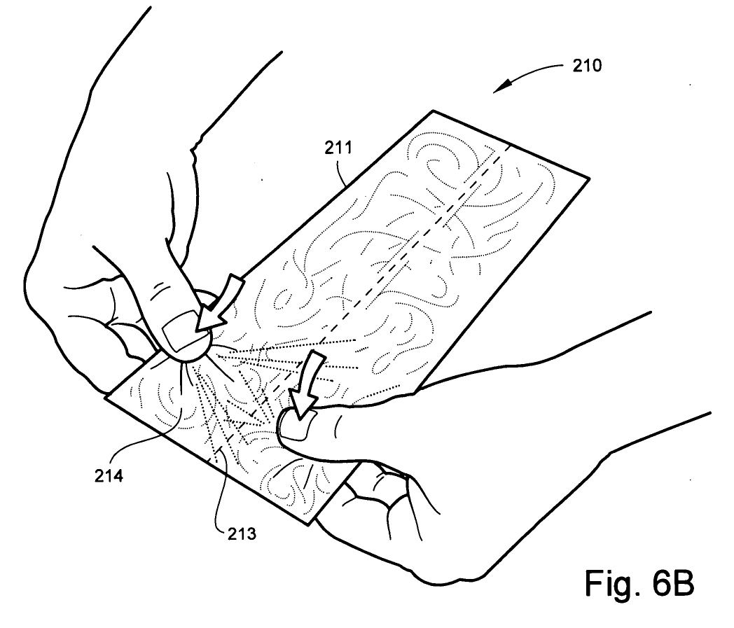

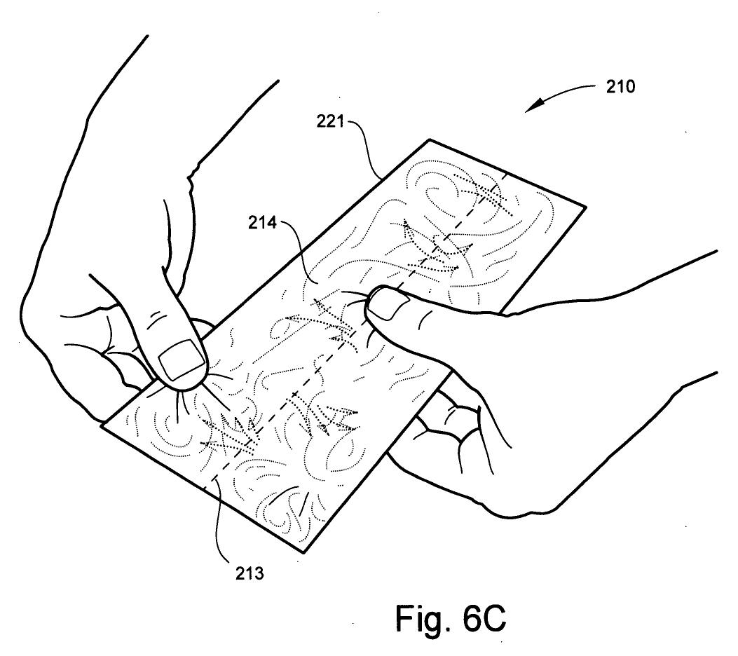

FIGS. 6A-6C show a splint 210 according to a third embodiment. In this splint 210, the pre-polymer is contained in an elongate envelope 214 equal to about half the size of an envelope 211. The envelope 214 is positioned along a top or bottom edge of the envelope 211. When a seal 213 of the envelope 214 is broken, the pre-polymer is mixed with the activator in the envelope 211.

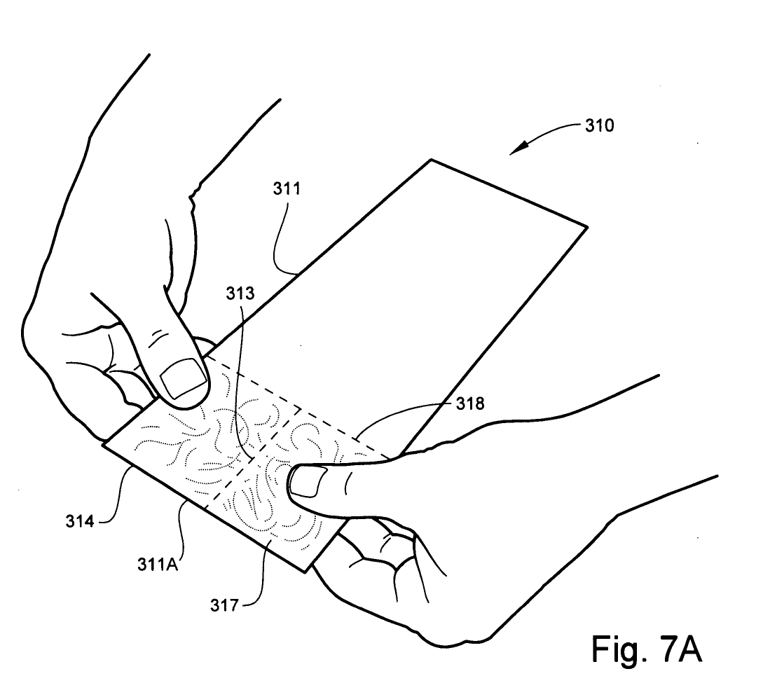

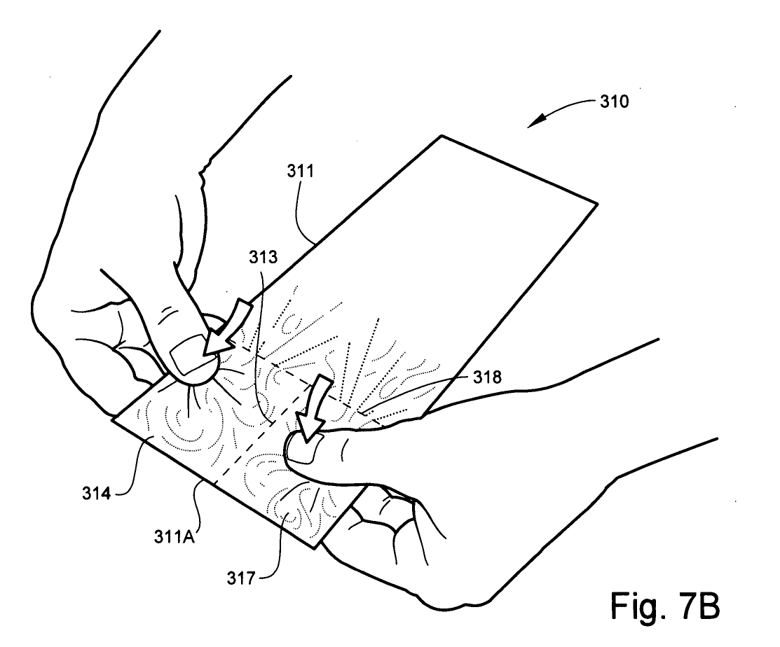

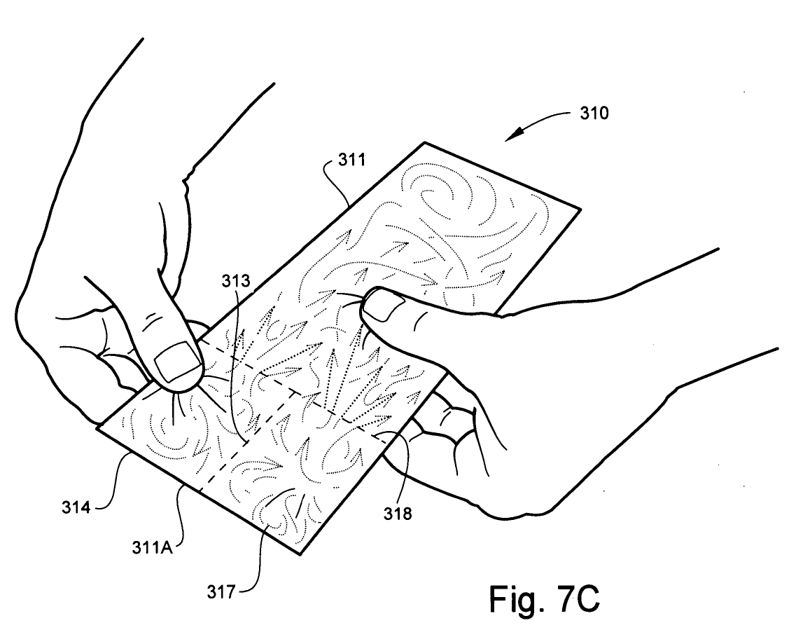

Referring now to FIGS. 7A-7C, a splint 310 configured with two envelopes 314 and 317 positioned on one end 311A of an envelope 311. The pre-polymer is contained in envelope 314, and the activator is contained in envelope 317. When seal 313 of envelopes 314 and 317 is broken, the pre-polymer and activator mix in the envelope 317 and the reaction breaks the frangible seal 318. Then the mixed polymer is spread across the length in envelope 311.

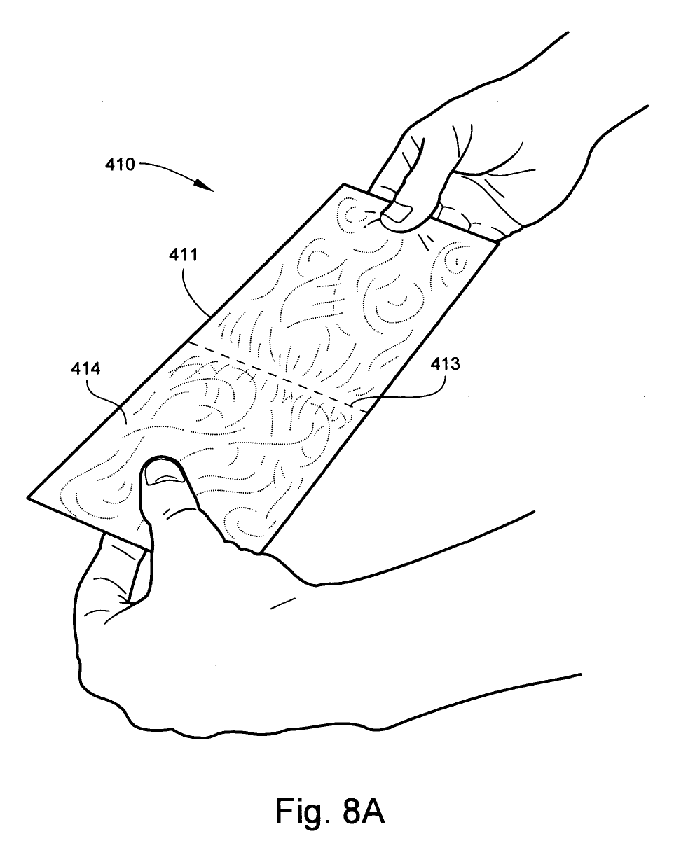

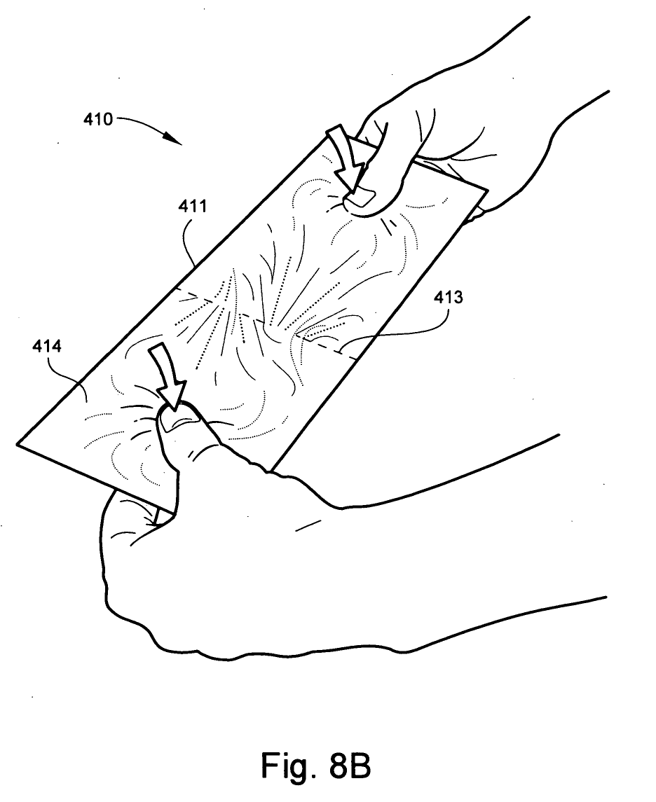

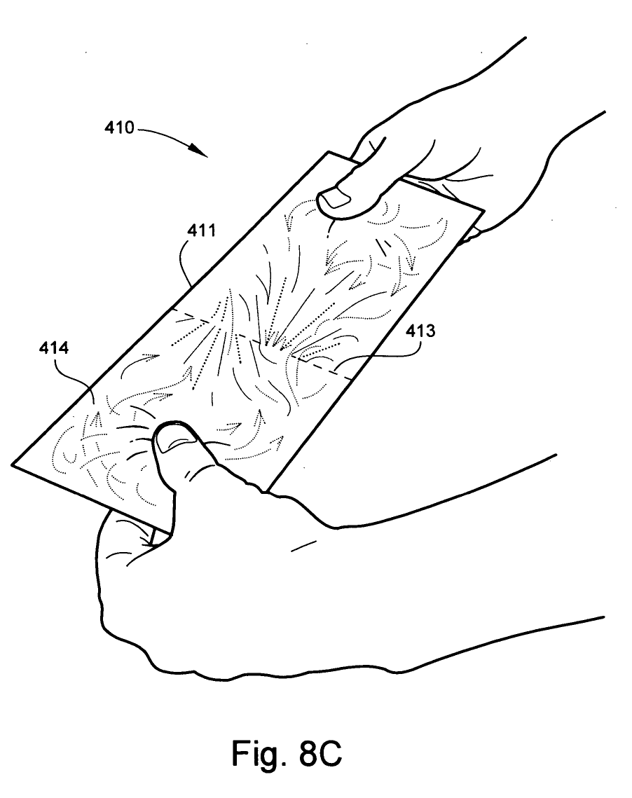

As illustrated in FIGS. 8A-8C, a splint 410 has a single envelope 414 equal to about half the size of an envelope 411. The envelope 414 is positioned on one end of the envelope 411 and extends to about the middle of the envelope 411. The envelope 414 contains the pre-polymer. When seal 413 of the envelope 414 is broken, the pre-polymer mixes with the activator in the envelope 411.

The envelope containing the pre-polymer may be formed as a portion of the envelope containing the activator, and initially separated by a weakness line or other means of keeping the components apart, such as a divider clip or a clamp or other such sealing mechanism.

Alternatively, the one envelope may be a separate structure inserted into the other envelope during manufacture, and which is ruptured by pressure applied through the wall of the other envelope, as illustrated in the sixth and seventh embodiments discussed below.

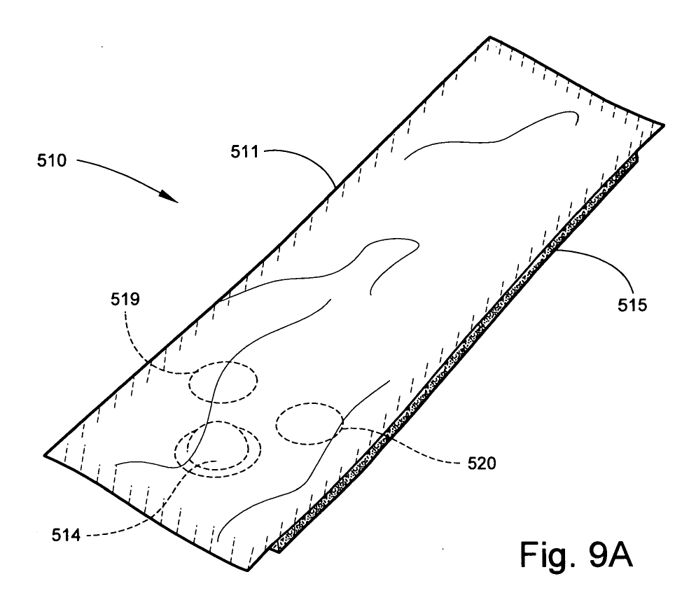

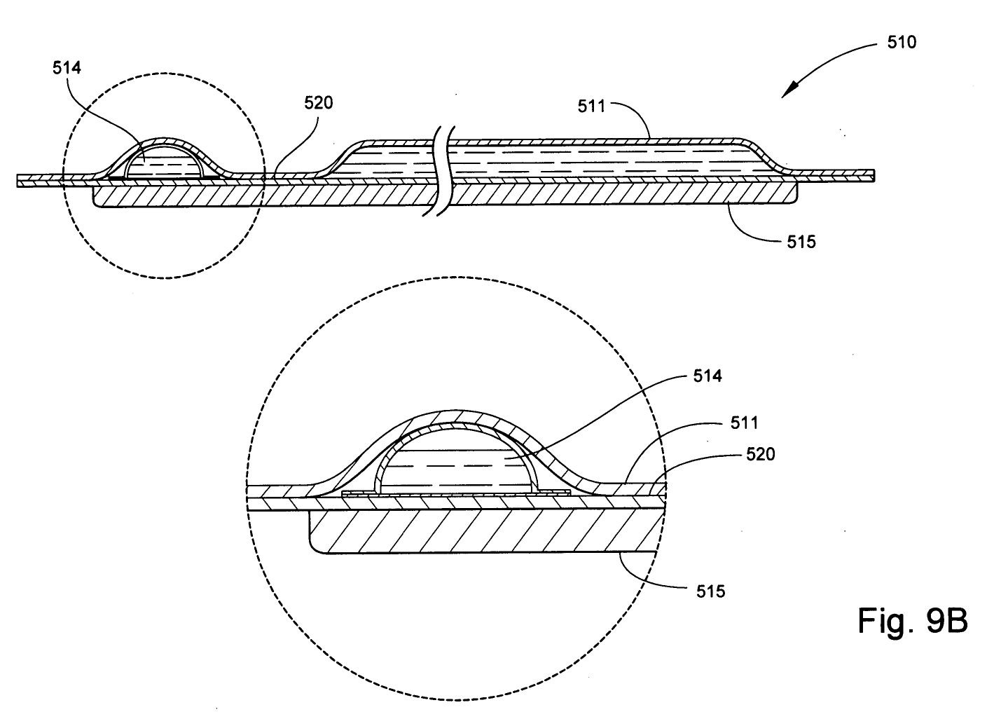

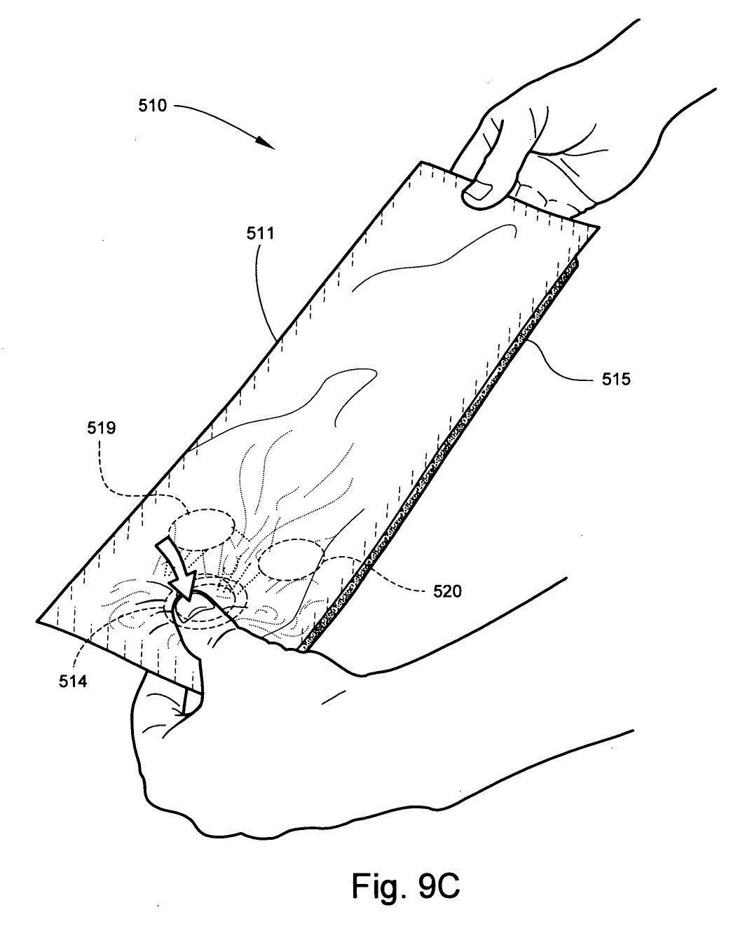

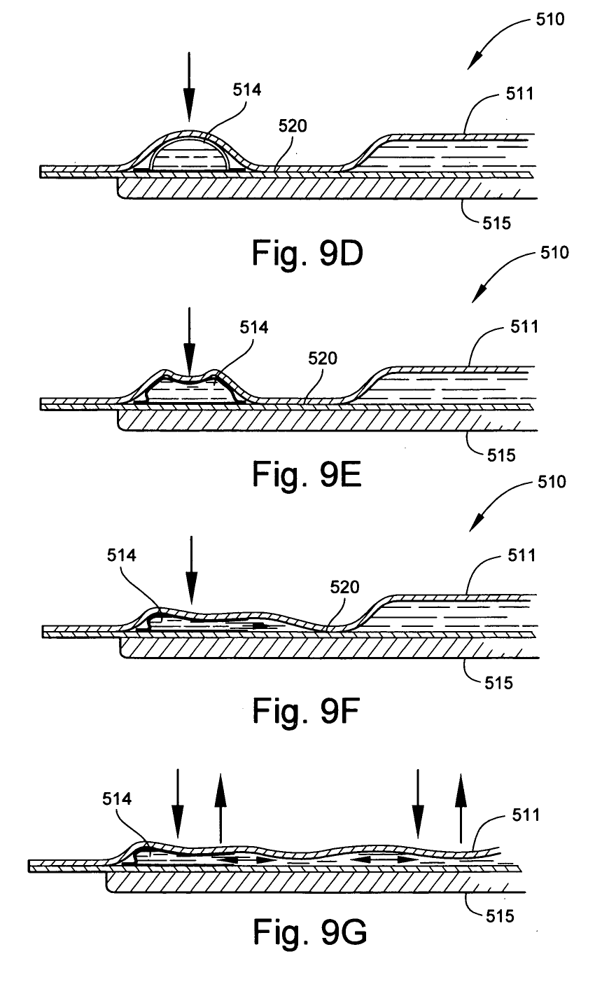

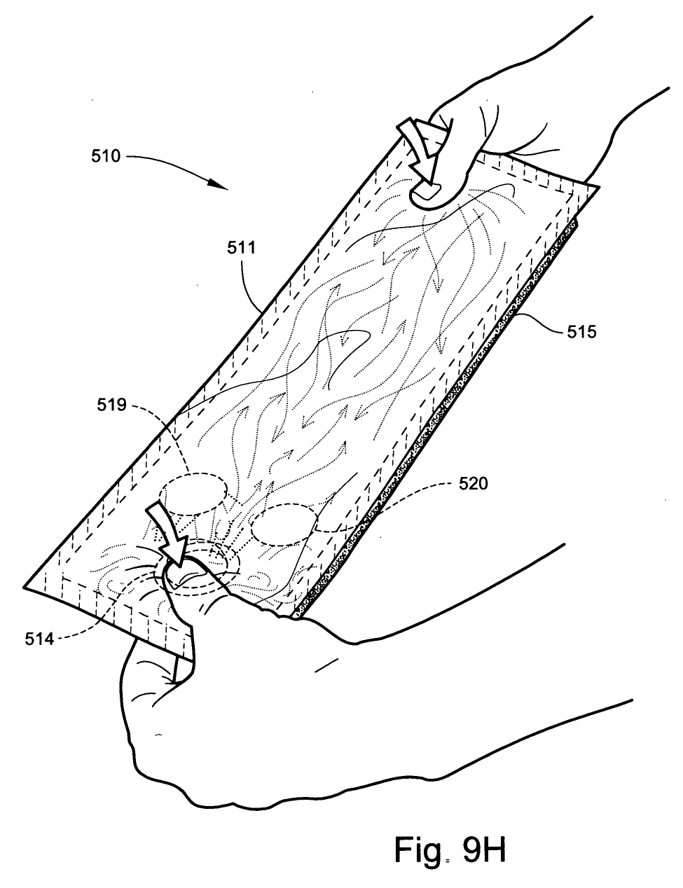

As is shown in FIGS. 9A-9H, splint 510 includes an envelope in the form of a burstable or rupturable bubble 514 is positioned inside an envelope 511 and held in position by a pair of annular seals 519 and 520. The bubble 514 may be constructed of any suitable material such as polyethylene, foil, PCTFE, polyester, or a combination thereof which allows the bubble 514 to contain one of the two components and rupture under manual pressure. The envelope 511 may contain one or more bubbles 514 depending on the amount of a respective component required for a particular splint size.

For purposes of clarity, the following discussion will be limited to the splint 510 having a bubble 514 containing a pre-polymer and an envelope 511 containing the activator. The splint 510 is activated by applying pressure to the bubble 514, illustrated in FIGS. 9C-9F, bursting the bubble, and releasing the pre-polymer into the envelope 511. This can be done by applying manual pressure on the bubble 514 or by rolling the splint 510 into a roll-like or similar form that will result in bursting the bubble 514. The pressure applied to the bubble 514 causes the bubble to burst, releasing the pre-polymer, Figure 9G, thereby allowing the pre-polymer to mix with the polyol in the envelope 511. Mixing is best achieved by alternately massaging the ends of the envelope 511, illustrated in FIG. 9H.

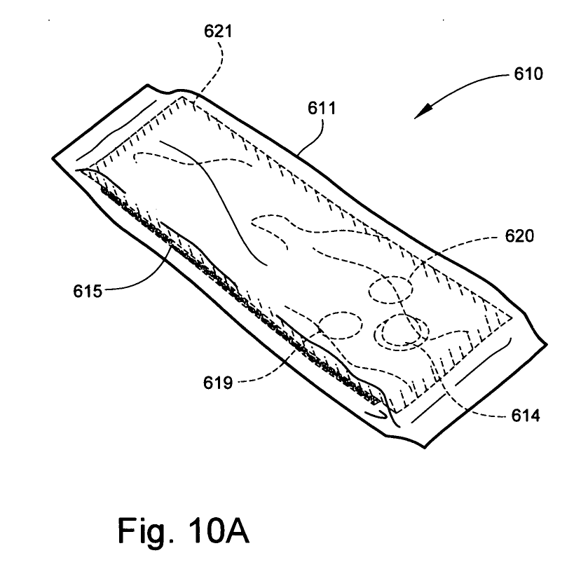

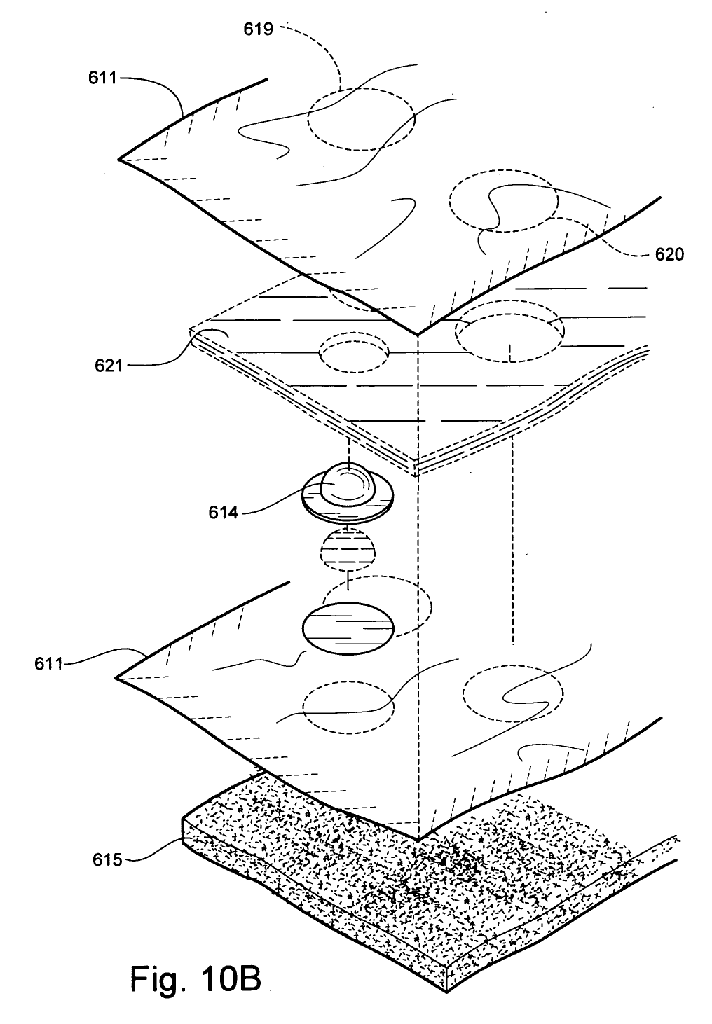

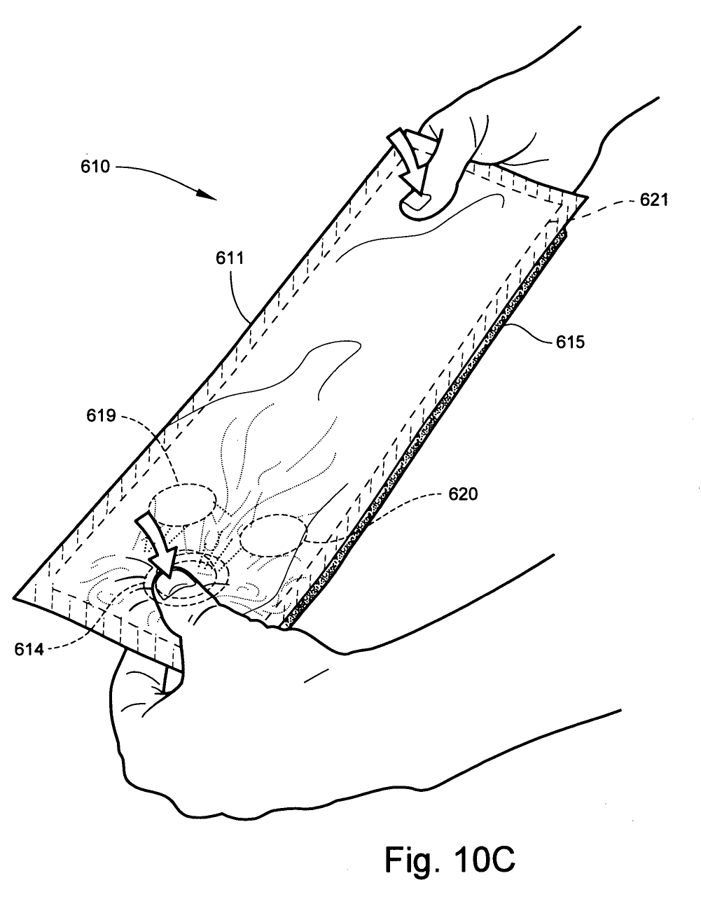

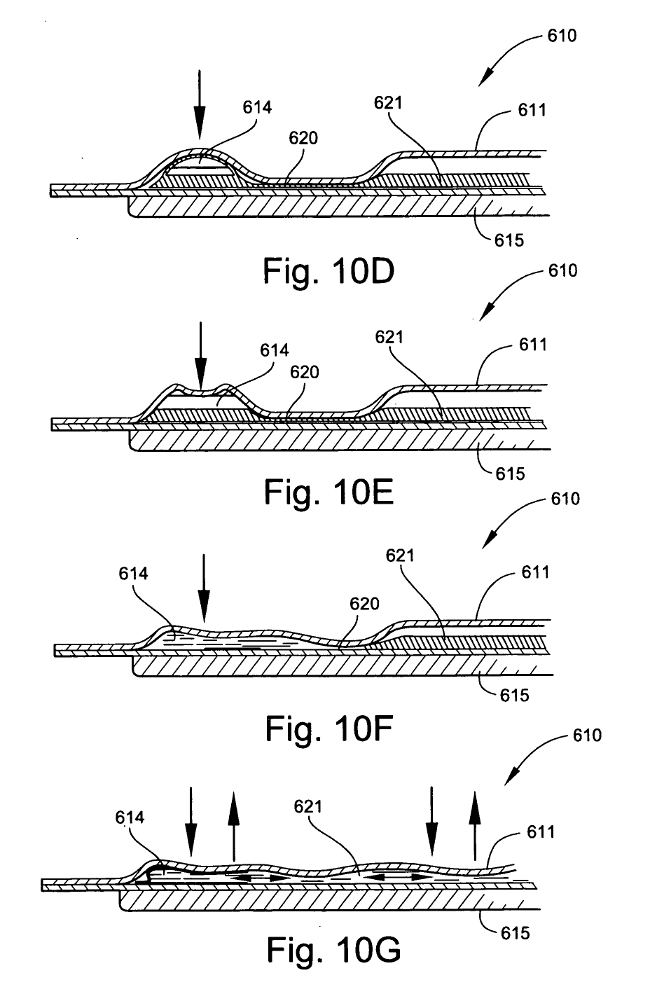

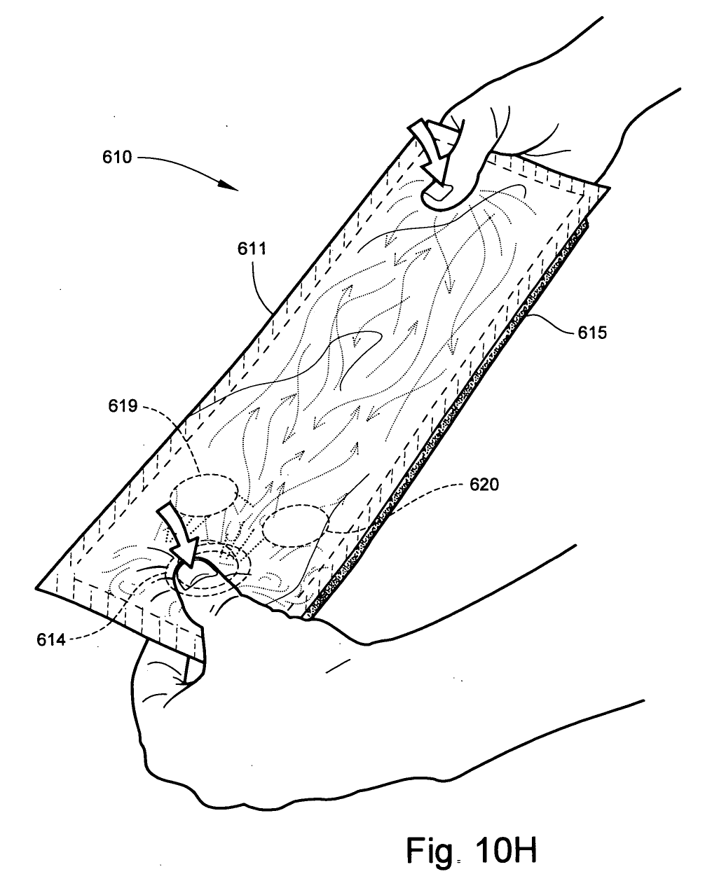

A splint 610 according to another embodiment is illustrated in FIGS. 10A-10H. In this embodiment, a substrate 621 made of a woven, non-woven, or knitted fabric is positioned within the interior of the envelope 611, and may be fixed in position by laminating or bonding across the length of the envelope 611. The substrate 621 may be constructed of any suitable organic or inorganic fiber such as polyethylene, polypropylene, para-aramid, and polyester, and is coated or impregnated with one of the two components used for forming the splint. Preferably, the substrate 621 is impregnated with the pre-polymer.

An envelope in the form of a burstable or rupturable bubble 614 is positioned within the envelope 611 and held in position by a pair of seals 619 and 620. The bubble 614 may contain either of the two components to keep the components in the envelope 611 from mixing. Preferably, the bubble 614 contains the activator.

For purposes of clarity, the following discussion will be directed to a splint 610 having a substrate 621 impregnated with a pre-polymer and a bubble 614 containing an activator. The splint 610 is activated by applying pressure to the bubble 614, illustrated in FIGS. 10D-10F, and bursting the bubble which releases the activator onto the impregnated substrate 621. The pressure applied to the bubble 614 causes the bubble to release the activator, FIG. 10G, thereby allowing the activator to mix with the pre-polymer.

Once released from the bubble 614, the activator is spread over the pre-polymer impregnated substrate, mixing the pre-polymer and activator together, and thereby forming a rigid polyurea/polyurethane structure within a few minutes. Mixing is best achieved by massaging across the whole length the ends of the envelope 611, illustrated in FIG. 10H, to distribute the activator evenly along the substrate 621.

The splint 610, illustrated in FIG. 10A, is manufactured in various pre-cut lengths, eliminating the need to cut the splint 610 to fit various body parts. For example, the splint 610 can be manufactured to fit the arm of a child or the leg of an adult male. Thus, various sized splints can be manufactured to fit various body parts of children and adults.

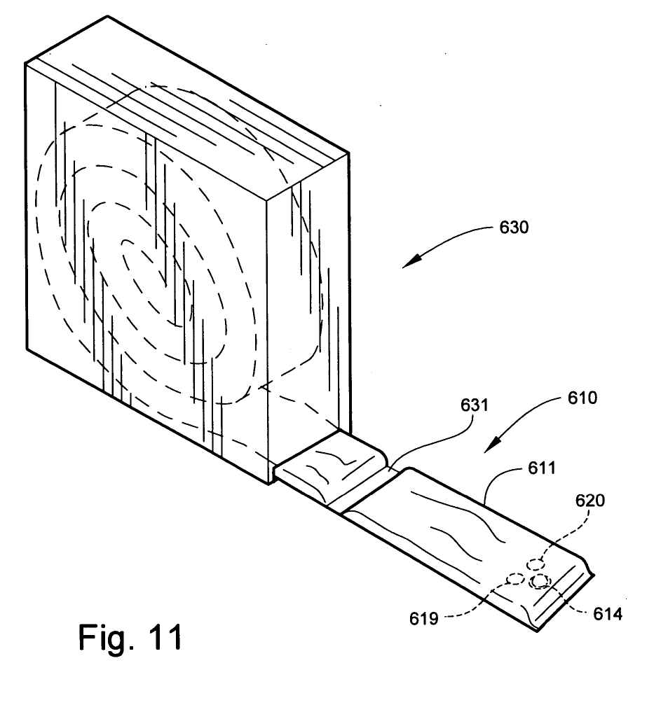

Referring to FIG. 11, each of the splints disclosed herein may be formed into a continuous roll 630. The continuous roll 630 gives the user the freedom to cut the splint to meet the exact need and requirements for each patient to ensure a better anatomical fit. The continuous roll 630 is formed of a plurality of splints 610, as illustrated, attached to each other end to end by a seal 631 so that the splints 610 may be separated without rupturing the envelope 611. Thus, a user can remove one or more splints 610 from the continuous roll 630 to obtain a desired length of splint 610 for a patient.

An orthopedic splint is described above. Various details of the invention may be changed without departing from its scope. Furthermore, the foregoing description of the preferred embodiments of the invention and the best mode for practicing the invention are provided for the purpose of illustration only and not for the purpose of limitation.

We claim:

1. An orthopedic splint, comprising:
(a) an initially flexible and conformable envelope containing a first component and a second component of a polymer system; and
(b) a separation means for separating the first component from the second component within the envelope, wherein actuation of the separation means allows the first component and the second component to mix in the envelope, thereby forming a hardened splint.

2. The orthopedic splint according to claim 1, wherein the envelope has a length sufficient to extend a length of a body part and a width sufficient to wrap approximately 50% of a circumference of the body part for immobilizing the body part while allowing for swelling of the body part.

3. The orthopedic splint according to claim 1, wherein the envelope is constructed of a material selected from the group consisting of aluminum foil, polyester, polypropylene, polyurethane, nylon, PCTFE, PVDC, metallised polyester, metallised polypropylene, PTFE, polyethylene, PVC, EVA, and a laminate formed using two or more of these materials.

4. The orthopedic splint according to claim 1, wherein the envelope includes a protective layer positioned on an outside surface of the envelope for being positioned between a body part and the splint and providing protection to a patient.

5. The orthopedic splint according to claim 1, wherein the first component is a pre-polymer and the second component is an activator.

6. The orthopedic splint according to claim 5, wherein the pre-polymer is selected from the group consisting of polyurethane, polyurea, epoxy, polyolefin, polyester, and silicone.

7. The orthopedic splint according to claim 5, wherein the activator is selected from the group consisting of water, hydrogel, amines, and polyols.

8. The orthopedic splint according to claim 1, and further including a substrate contained within the envelope, the substrate being coated or impregnated with a respective one of the first and second components.

9. The orthopedic splint according to claim 1, wherein the splint has a predetermined pre-cut length for being applied to a body part.

10. The orthopedic splint according to claim 1, wherein the splint is in roll form for being dispensed in lengths suitable for a given medical use.

11. An orthopedic splint, comprising:
(a) a first polymer system component contained within a first envelope;
(b) a second polymer system component contained within a second envelope; and
(c) the second envelope is positioned within the first envelope to thereby position the first and second polymer system components within the first envelope in an initially unmixed, flexible and conformable state, wherein the second envelope is rupturable to allow the first and second polymer system components to mix within the first envelope, forming a hardened splint.

12. The orthopedic splint according to claim 11, wherein the envelope is constructed of a material selected from the group consisting of aluminum foil, polyester, polypropylene, polyurethane, nylon, PCTFE, PVDC, metallised polyester, metallised polypropylene, PTFE, PVC, EVA, polyethylene and blends thereof.

13. The orthopedic splint according to claim 11, wherein the first component is a pre-polymer selected from the group consisting of polyurethane, polyurea, epoxy, polyolefin, polyester, and silicone.

14. The orthopedic splint according to claim 11, wherein the second component is an activator selected from the group consisting of water, hydrogel, amines, and polyols.

15. The orthopedic splint according to claim 11, wherein the second envelope is a part of the first envelope and is defined by and separated from the first envelope by a seal.

16. The orthopedic splint according to claim 11, wherein the second envelope is a separate structure inserted into the first envelope.

17. The orthopedic splint according to claim 11, wherein the splint has a predetermined pre-cut length for being applied to a body part.

18. The orthopedic splint according to claim 11, wherein the splint is in roll form for being dispensed in lengths suitable for a given medical use.

19. The orthopedic splint according to claim 11, and further including an initially flexible and conformable substrate contained within the first envelope, the substrate being impregnated with a respective one of the first and second components.

20. The orthopedic splint according to claim 19, wherein the substrate is a fabric constructed of a fiber selected from the group consisting of polyethylene, polypropylene, para-aramid, and polyester.

21. An orthopedic splint, comprising:
(a) an initially flexible and conformable envelope for containing a polymer system, the polymer system comprising:
(i) a, pre-polymer;
(ii) a activator for mixing with the pre-polymer to form a hardened splint;
(b) a substrate contained within the envelope and impregnated with the pre-polymer; and
(c) at least one burstable bubble positioned within the envelope for containing the activator, thereby maintaining the pre-polymer and polyol within the envelope in an unmixed state, wherein the at least one burstable bubble is rupturable to allow the activator to mix with the pre-polymer contained within the substrate, forming the hardened splint.

22. The orthopedic splint according to claim 21, and further including seals to hold the at least one burstable bubble in a desired position within the envelope.

23. The orthopedic splint according to claim 21, wherein the at least one burstable bubble is constructed of a material selected from the group consisting of polyethylene, foil, PCTFE, polyester, polypropylene, and nylon.

24. The orthopedic splint according to claim 21, wherein the splint has a predetermined pre-cut length for being applied to a body part.

25. The orthopedic splint according to claim 21, wherein the splint is in roll form for being dispensed in lengths suitable for a given medical use.

26. A method of constructing an orthopedic splint comprising the steps of:
(a) providing an initially flexible and conformable envelope; and
(b) forming first and second compartments in the envelope; and
(c) introducing a first component into the first compartment and a second component into the second compartment, so that when mixed a hardened splint is formed.

27. The method according to claim 26, and further comprising the step of applying a protective layer on an outside surface of the envelope.

28. A method of immobilizing a body part, comprising the steps of:
(a) providing an orthopedic splint, comprising an initially flexible and conformable envelope containing a first component and a second component of a polymer system, wherein the first and second component are maintained separate from each other;
(b) manipulating the envelope to allow the first component and the second component to mix;
(c) placing the splint into engagement with the body part; and
(d) securing the splint to the body part in a closely-conforming configuration for a period of time sufficient to allow the splint to harden.

29. The method according to claim 28, and further including the step of massaging opposing ends of the splint to provide mixing of the first and second components.

30. The method according to claim 28, and further including the step of molding the splint to the body part while the splint is flexible.
